(19) United States
(12) Patent Application Publication
(10) Pub. No.: US 2025/0303152 A1
(43) Pub. Date: Oct. 2, 2025

(54) INTRACELLULAR TREATMENT DEVICE AND METHODS OF USE THEREOF

(71) Applicant: Vector Surgical, LLC, Waukesha, WI (US)

(72) Inventor: Amanda Katsma, West Bend, WI (US)

(21) Appl. No.: 18/863,082

(22) PCT Filed: May 9, 2023
(86) PCT No.: PCT/US2023/021427

§ 371 Date: Nov. 5, 2024

Related U.S. Application Data
(60) Provisional application No. 63/364,750, filed on May 16, 2022.

(51) Int. Cl. A61N 1/32; A61M 5/145; A61M 5/168; A61N 1/05

(52) U.S. Cl. CPC A61N 1/327; A61N 1/0504; A61M 5/1452; A61M 5/16804

(57) ABSTRACT

The present disclosure relates to an intracellular device for delivering therapeutics. The device may be an in vivo electroporation device which may include at least one electrode needle adapted to produce an electric field and a plurality of infusion ports on at least one axially extending line along the length of the needle configured to deliver a therapeutic substance. The device may include a retractable insulating sheath configured to limit a treatment field of the device, and a penetration limiting means configured to limit the depth of insertion of the electrode needle. The device may include an injection needle configured to deliver a therapeutic substance and at least two electrode needles adapted to produce an electric field, which may cause poration of the target tissue's cells for uptake of the therapeutic substance.

### FIELD

The present disclosure generally relates to a device for a percutaneous delivery of intracellular therapies to tissue by electroporation needle system to improve the delivery and uptake of therapeutic agents in tissue.

### BACKGROUND

In vivo therapies have been proven to be effective to treat diseases that have been otherwise uncurable. To reach therapeutic effect, however, these therapies are often delivered systemically with very high vector doses which can trigger adverse immune responses. In addition, use of gene therapies are usually prohibitively expensive. Furthermore, high doses delivered systemically increase the risks of off-target effects, inflammation, and insertional mutagenesis.

Effective delivery of intracellular therapies may help millions of patients suffering from cancer and genetic disorders and may be used to cure diseases, such as diabetes, hemophilia, and sickle cell anemia, that otherwise require continuous, expensive treatments throughout a patient's life.

Systemic injection, surgical access and drug delivery catheters have associated risk and/or expense. No current method offers facilitative effects to directly access the target tissue overcoming the immune response, the vessel wall endothelial barrier, or create an adjustable field of treatment. Use of exosomes, nanoparticles or other means may also aid in delivery but are many years away from clinical application.

Application of electrical signals in tissue relative to the administration of a prophylactic or therapeutic agent can have desirable effects on uptake of the agent by the tissue and/or the agent to be delivered. Specifically, techniques, such as electroporation and iontophoresis, may be utilized to significantly improve the delivery and/or uptake of a variety of agents in tissue.

In spite of the promise associated with electrically mediated agent delivery and the potential clinical applications of these techniques, progress has been hampered by the lack of an effective means to achieve the overall objective of efficient and reliable agent delivery using these techniques. One of the most significant shortcomings of current systems is the inability to achieve reliable and consistent application from subject to subject. Significant sources of this variability are due to differences in the technique and skill level of the operator and the inability to accurately and consistently target treatment fields. Other sources of variability that are not addressed by current systems include differences in the physiologic characteristics between patients that can affect the application of the procedure.

Accordingly, there is a need for a drug delivery device that is capable of delivering therapy directly to the target tissue through percutaneous access which addresses the consistency and ease of use issues discussed above.

### SUMMARY

An in vivo electroporation device is disclosed including a housing comprising at least one electrode needle comprising a penetration tip; and a shaft that is elongated, hollow, and adapted for delivery of a therapeutic substance to a tissue, said shaft having a length running from one end of the needle to the penetration tip of the needle at the other end of the needle, wherein the shaft comprises a charged electrode adapted to produce an electric field; and a plurality of infusion ports on at least one axially extending line along the length; a retractable insulating sheath configured to limit a treatment field of the device; and a penetration limiting means configured to limit the depth of insertion of the electrode needle.

An in vivo electroporation device is disclosed, where the device includes a housing comprising an injection needle comprising a penetration tip, and a shaft that is elongated, hollow, and adapted for delivery of a therapeutic substance to a tissue, said shaft having a length running from a proximal end of the injection needle to the tip of the injection needle at a distal end of the needle, wherein the shaft comprises a plurality of infusion ports, at least two electrode needles, each electrode needle comprising a penetration tip, a shaft having a length running from a proximal end of the electrode needle to the tip of the electrode needle at a distal end of the needle and comprising at least one electrode adapted to produce an electric field, and an insulating sheath surrounding the shaft and configured to move relative to the shaft between an extended position covering the distal end of the shaft and a retracted position exposing the distal end of the shaft, and a penetration limiting means for controlling the depth of insertion of the at least two electrode needles and the injection needle.

A method of treating a disease in a patient in need thereof is disclosed including inserting a device comprising a therapeutic substance disposed therein into a tissue of the patient in vivo. The device may include a housing comprising at least one electrode needle comprising a penetration tip and a shaft that is elongated, hollow, and adapted for delivery of the therapeutic substance to a tissue, said shaft having a length running from one end of the needle to the penetration tip of the needle at the other end of the needle, wherein the shaft comprises a charged electrode adapted to produce an electric field, and a plurality of infusion ports on at least one axially extending line along the length, a retractable insulating sheath configured to limit a treatment field of the device, and a penetration limiting means configured to limit the depth of insertion of the electrode needle, and activating an electrical energy source in electrical communication with each of the at least one electrode needle, thereby providing a pulse of electrical energy to the at least one electrode causing poration of the target tissue's cells for their uptake of the therapeutic substance.

In another embodiment, the method includes inserting a device comprising a therapeutic substance disposed therein into a tissue of the patient in vivo, wherein the device further comprises a housing comprising an injection needle comprising a penetration tip, and a shaft that is elongated, hollow, and adapted for delivery of a therapeutic substance to a tissue, said shaft having a length running from a proximal end of the injection needle to the tip of the injection needle at a distal end of the needle, wherein the shaft comprises a plurality of infusion ports, at least two electrode needles, each electrode needle comprising a penetration tip, a shaft having a length running from a proximal end of the electrode needle to the tip of the electrode needle at a distal end of the needle and comprising at least one electrode adapted to produce an electric field, and an insulating sheath surrounding the shaft and configured to move relative to the shaft between an extended position covering the distal end of the shaft and a retracted position exposing the distal end of the shaft; and a penetration limiting means for controlling the depth of insertion of the at least two electrode needles and the injection needle, and activating an electrical energy source in electrical communication with each of the at least two electrodes, thereby providing a pulse of electrical energy to the at least two electrode causing poration of the target tissue's cells for their uptake of the therapeutic substance.

### BRIEF DESCRIPTION OF THE DRAWINGS

The accompanying drawings, which are incorporated herein and constitute part of this specification, are illustrative of particular embodiments of the present disclosure and do not limit the scope of the present disclosure.

FIG. 1 is a drawing illustrating an in vivo electroporation device having two electrode needles and one injection needle.

FIG. 2 is a drawing illustrating an in vivo electroporation device having an electrode needle that also functions as an injection needle.

FIG. 3 is a drawing illustrating an in vivo electroporation device having two electrode needles having infusion ports on an axially extending line facing the other needle.

FIG. 4A is a drawing illustrating a top view of an in vivo electroporation device having two electrode needles.

FIG. 4B is a drawing illustrating a side view of the in vivo electroporation device having two electrode needles in FIG. 4A.

FIG. 5 is a drawing illustrating a distal cross-sectional view of an insulating sheath covering the electrode needles.

FIG. 6A is a drawing of a top cross-sectional view of the device in FIG. 4A showing the housing, the penetration lock, the penetration limiting means, and the electrode needles.

FIG. 6B is a drawing of a top cross-sectional and close up view of the penetration limiting means of FIG. 6A, including three slides that attach the penetration limiting means to the housing.

FIG. 6C is a drawing of a distal cross-sectional view of the penetration limiting means of FIG. 6B, having two holes where the electrodes pass through.

FIG. 6D is a drawing of a proximal cross-sectional view of the penetration limiting means of FIG. 6B, showing the three slides and two holes where the electrodes pass through.

FIG. 7 is a drawing of the movement of electric current using the electrode needles as charged electrodes.

FIG. 8A is a drawing of a top perspective view of the insulating sheath and handle to slide the insulating sheath into place.

FIG. 8B is a drawing of a proximal cross-sectional view of the insulating sheath of FIG. 8A showing the passage of the two electrode needles, the penetration lock, and the cathode and anode electrodes.

FIG. 9A is a drawing of a proximal cross-sectional view of a housing of an in vivo electroporation device including a port for attaching tubing or an infusion means and, below, an electrical connection/cord plug-in.

FIG. 9B is a drawing of a top perspective view of the housing of FIG. 9A, including an opening for the handle for the insulating sheath and openings for the electrodes, penetration limiting means, and insulating sheath at the distal end.

### DETAILED DESCRIPTION

The device disclosed herein may be used clinically, for example, in the delivery of chemotherapeutic drugs and/or therapeutic genes in tumors, the delivery of DNA vaccines for prophylactic and therapeutic immunization, and the delivery of nucleic acid sequences encoding therapeutic proteins.

This disclosure is directed to an intracellular device for delivering therapeutics directly to a target tissue. The device may facilitate electrically mediated technologies such as electroosmosis, electrophoresis, and electropermeabilization in addition to electroporation.

The in vivo electroporation device of the disclosure herein may include a housing comprising at least one electrode needle, a retractable insulating sheath configured to limit a treatment field of the device, and a penetration limiting means configured to limit the depth of insertion of the electrode needle. The electrode needle may include a penetration tip and a shaft that is elongated and hollow, the shaft having a length running from one end of the needle to the penetration tip of the needle at the other end of the needle. The shaft of the electrode needle may comprise a charged electrode adapted to produce an electric field. The shaft of the electrode needle may also include a plurality of infusion ports on at least one axially extending line along the length, such that the electrode needle may be adapted for delivery of a therapeutic substance to a tissue. The device may further include an infusion chamber to hold the therapeutic substance before administration. At least a portion of the infusion chamber may be in the shaft, for example, along the length of the shaft. When the device includes two electrode needles, the infusion ports on one electrode needle may face the infusion ports on the opposite electrode needle.

The device described herein offers percutaneous delivery directly to tissues (skin, muscle, organ parenchyma) via a simple skin puncture. It may be used in conjunction with ultrasound guided internal navigation allowing for injection of therapy directly into the target tissue avoiding the obstacles and dangers of systemic delivery and vessel wall crossing. Once the needle(s) has reached its target and the treatment is injected, the integrated electrodes may be configured to emit low level electrical pulses creating a reversible electroporation field facilitating treatment transfection. The configuration of the device disclosed herein may allow for a higher transfection rate and a lower dose amount.

Therapeutic substances contemplated for delivery using the device disclosed herein may include pharmaceuticals, proteins, nucleic acids, or a combination thereof. The term “therapeutic substance” will be used herein in its broadest sense to include any agent capable of providing a desired or beneficial effect on living tissue. Thus, the term will include both prophylactic and therapeutic agents, as well as any other category of agent having such desired effects. Therapeutic agents include, but are not limited to pharmaceutical drugs and vaccines, and nucleic acid sequences (such as supercoiled, relaxed, and linear plasmid DNA, antisense constructs, artificial chromosomes, or any other nucleic acid-based therapeutic), and any formulations thereof. Such agent formulations include, but are not limited to, cationic lipids, cationic polymers, liposomes, saline, nuclease inhibitors, anesthetics, poloxamers, preservatives, sodium phosphate solutions, or other compounds that can improve the administration, stability, and/or effect of the therapeutic agent.

The technique of electroporation utilizes the application of electric fields to induce a transient increase in cell membrane permeability and to move charged particles. By permeabilizing the cell membranes within the target tissue, electroporation dramatically improves the intracellular uptake of exogenous substances that have been administered to the target tissue. The increase in cell membrane permeability and molecular movement due to electroporation offers a method for overcoming the cell membrane as a barrier to therapeutic agent delivery. The physical nature of the electroporation technique allows it to be applied in virtually all tissue types.

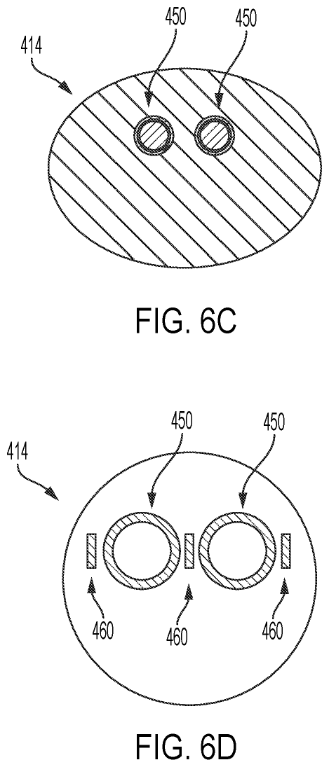

The device may include a fixed electrode array with adjustable electrode length. The device may include a penetration limiting means configured to limit the depth of insertion of the electrode needle(s). The penetration limiting means may comprise a penetration limiting structure operatively connected to the housing to adjust the length of the electrode needle(s). The penetration limiting means may comprise a penetration limiting lock to lock the penetration limiting means in place while the device is in use. For example, the lock may comprise a push button lock, a sliding lock, a switch toggle lock, a combination thereof, or the like. The penetration limiting means may adjustable. In the non-limiting example depicted in FIG. 6A-6D, a penetration limiting means 414 may have two holes 450 where the electrode needles 422, 421 pass through. The penetration limiting means may include a plurality of slides proximal and distal along the electrodes and can be locked into place, for example, with a penetration limiting lock 420, that may extend axially outward from the housing. Three slides 460 are shown that attach the penetration limiting means to the device housing 412.

The charged electrode may comprise at least two monopolar electrodes, wherein the at least two monopolar electrodes are spaced and electrically isolated from one another. The fixed electrode needles allow for parallel electrode positioning and standardized distance for optimizing the therapeutic field. The charged electrode may include a bipolar electrode. The charged electrode may run an entire length of the shaft. The charged electrode may comprise a conductive material. The conductive material may comprise any conductive metal, such as stainless steel, platinum, or the like, or a combination thereof. The conductive material may be plated with silver, gold, aluminum, or a combination thereof.

The insulating sheath may be retractable. The insulating sheath may surround a shaft of an electrode needle at least in part, or in entirety. For example, the insulating sheath may be at least in part cylindrical in shape to surround an electrode needle. The insulating sheath may include a distal end which is cylindrical in shape and surrounds the shaft of the electrode needle(s). The proximal end of the insulating sheath may include a handle to slide the insulating sheath or other handle configured to position the insulating sheath on the electrode needle to limit the treatment field. The insulating sheath may comprise cut-outs for attachment of electrodes.

The insulating sheath may comprise a material that insulates against electrical current as well as seals the infusion ports and can be visualized on imaging, such as ultrasound. The insulating sheath may be extended over the shaft of the electrode needle using an actuator. For example, the actuator may be a button or handle used to slide the insulating sheath into position. The button or handle may be operatively coupled with the insulating sheath and may be configured to allow for the insulating sheath to travel along a sheath slide track. The button or handle may allow for single hand operation of the device. At a proximal end, the insulating sheath may include a plurality of apertures designed to allow for the passage of the two electrode needles, and subsequently, the integrated cathode and anode electrodes. The insulating sheath may be composed of a single piece of non-conductive, somewhat pliable material.

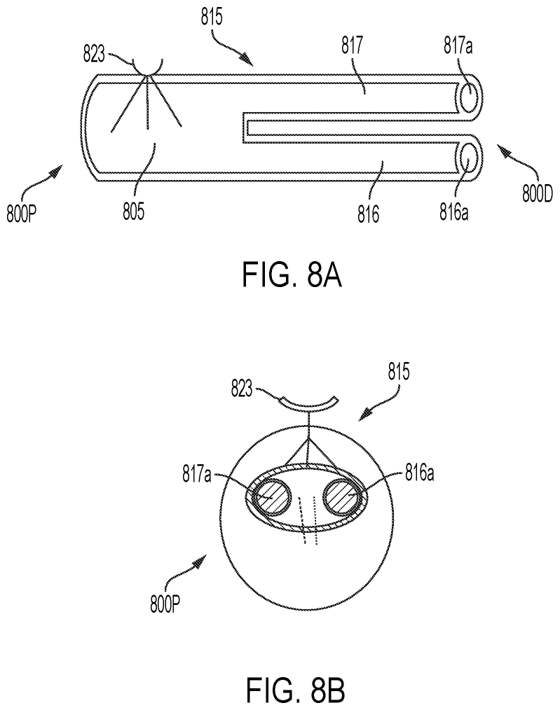

Referring to the example in FIG. 8A and 8B, an insulating sheath 815 may be extended using a handle 823 to slide the insulating sheath into place. The insulating sheath 815 is oval shaped at a proximal end 800P with a single lumen 805 that splits into two cylinders 816, 817 distally. The cylinders 816, 817 may include apertures 816a and 817a at the distal end 800D of each, and be designed to encircle the electrode needles and may slide in and out of the housing to cover more or less of the distal electrode needles, thereby increasing or decreasing the treatment field size.

The insulating sheath may provide a snug fit insulating the current and sealing shut the infusion ports. Materials for use with the insulating sheath comprise vinyl (such as polyvinyl chloride), red rubber latex, and silicone rubber polymer. There may be a handle attached within the housing to the proximal, oval shaped aspect of the insulating sheath that extends through a slot in the housing to the outer aspect allowing the user to adjust the position of the sheath relative to the electrode needles.

The total length of the insulating sheath may be about 100 mm to about 300 mm, about 100 mm to about 275 mm, about 100 mm to about 250 mm, about 100 mm to about 230 mm, about 100 mm to about 220 mm, about 150 mm to about 300 mm, about 175 mm to about 300 mm, about 200 mm to about 300 mm, or a value within any of these ranges. For example, the insulating sheath may be about 216 mm in total length.

The cylinders of the insulating sheath may be about 50 mm to about 250 mm in length, such as about 75 mm to about 250 mm, about 100 mm to about 250 mm, about 125 mm to about 250 mm, about 150 mm to about 250 mm, about 50 mm to about 225 mm, about 50 mm to about 200 mm, about 50 mm to about 175 mm, or a value within any of these ranges. For example, he cylinders of the insulating sheath may be about 155 mm in length.

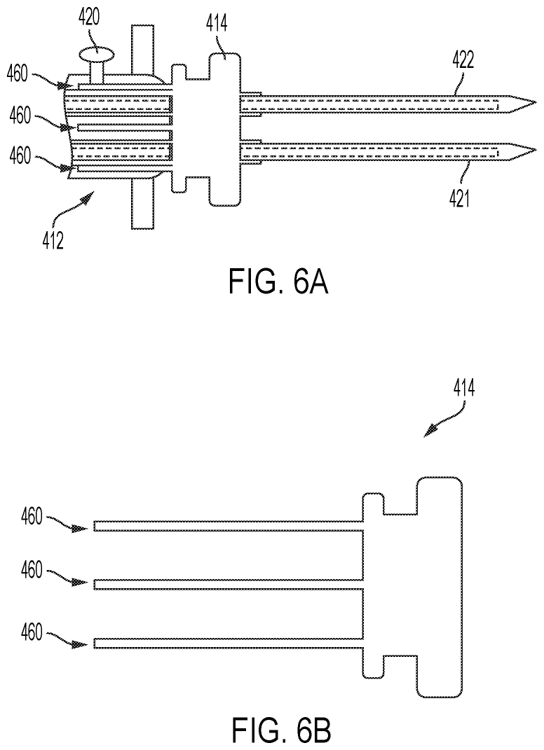

Referring to FIG. 5, the insulating sheath may cover the electrode needle such that in some instances, the electrode needle may be sealed and insulated. Where an infusion port is present and the insulating sheath is over the electrode needle, the port may be sealed and insulated. Where no insulating sheath covers the electrode needle, the electrode needle may be uninsulated and the infusion ports may be open.

Use of the device disclosed herein may allow access to peripheral organ tissue, such as access to all anterior aspects of the liver. Controlled speed of injection into the solid organ parenchyma is intended to create a temporary increase in pressure surrounding the tissue (i.e. hydrodynamic pressure) which further facilitates uptake of the therapeutic substance by cells. The device of embodiments herein allows for direct tissue access and avoids the circulatory system and other body barriers. By avoiding the circulatory system, the device may minimize direct contact of the therapeutic substance with blood/immune elements to minimize adverse reactions and uptake of the therapeutic substance by non-target cells, hypersensitivity, and neutralizing antibodies. Since the device disclosed herein may be used for percutaneous procedures, use of the device may be at an inpatient setting or an outpatient setting. The device disclosed herein may be used while the patient is under local anesthesia.

The housing of the in vivo electroporation device may further include a cathode attachment bracket and an anode attachment bracket. The housing may further include a port for the fluid delivery means, such as an infusion pump. The infusion pump may be at the proximal end of the housing. The device may further include a plug-in cord for the electroporation generator. Once the electrode needle(s) reaches its target and the therapeutic substance is injected, the integrated electrodes emit low level electrical pulses creating a reversible electroporation field facilitating treatment transfection allowing for a higher transfection rate and a lower dose amount.

The disclosure omits or only briefly describes conventional features of the disclosed technology that are apparent to those skilled in the art. Reference to various embodiments does not limit the scope of the claims attached hereto. Additionally, any examples and figures set forth in this specification are intended to be non-limiting and merely set forth some of the many possible embodiments for the disclosure. Further, particular features described herein can be used in combination with other described features in each of the various possible combinations and permutations. A person of ordinary skill in the art would know how to use the instant invention, in combination with routine experiments, to achieve other outcomes not specifically disclosed in the examples or the embodiments.

It is also to be understood that the terminology used in the description is for the purpose of describing the particular versions or embodiments only and is not intended to limit the scope of the present disclosure.

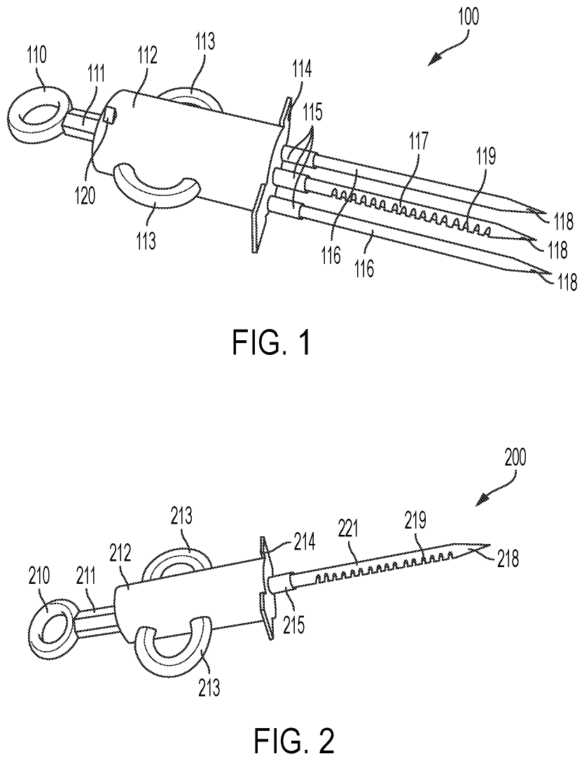

Referring to FIG. 1,, the in vivo electroporation device 100 may comprise a housing 112 comprising an injection needle 117 in the middle and two electrode needles 116 on either side of the injection needle 117. The housing may further include an injector 111. The injector 111 may include an injector loop 110, for ease of use such as for one handed operation of the device. Similarly the housing may also include loops 113 (or handles) to allow for better grip, including for one handed operation of the device. The device 100 may also include an insulating sheath 115 on each electrode needle 116 and injection needle 117 that can be deployed and retracted to adjust the treatment field for the individual patient and their anatomy. The housing may be operatively connected to a penetration limiting means 114 which may be controlled by a penetration limiting lock 120.

Ultrasound, or other imaging guidance, may be used to insert the device while avoiding damage to larger vessels or anatomic structures. Prior to percutaneous insertion of the device 100 into the patient, the penetration limiting means 114 may be locked using the penetration limiting lock 120 situated on the housing 112 at a predetermined maximum depth, protecting vital internal structures. The device may be inserted into the subject using the penetration tip 118 of the electrode needles 116 and the injection needle 117. The use of the injector loop 110 and housing loops 113 may allow for single hand operation of the handheld device while the subject is free to use imaging guidance, such as ultrasound, to guide the electroporation device 100 into position within the patient. Once the targeted tissue is reached, the insulating sheath 115 may be adjusted to fit the treatment field to the needs of the patient while avoiding treatment of unintended tissue. The injection of the therapeutic substance may be administered using the injector 111 through the plurality of infusion ports 119 of the injection needle 117. The treatment field may be pulsed with the electrodes connected to the electrode needles 116 facilitating uptake of the therapeutic substance.

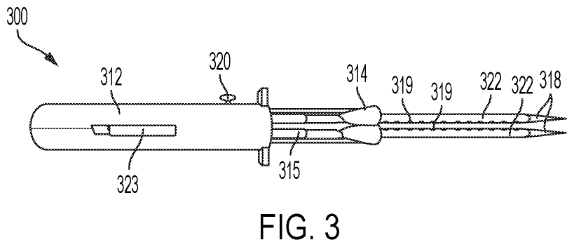

Referring to FIG. 2, the in vivo electroporation device 200 may comprise a housing 212 comprising a single electrode needle 221. The electrode needle 221 comprises a hollow shaft which includes an infusion chamber therein (not shown), and a plurality of infusion ports 219 along one or more axially extending lines on the electrode needle 221. The device may be inserted into the subject using the penetration tip 218 of the electrode needle 221. The housing may further include an injector 211. The injection of the therapeutic substance may be administered using the injector 211 through the plurality of infusion ports 219 of the electrode needle 221. The injector 211 may include an injector loop 210, for ease of use such as for one handed operation of the device. Similarly the housing may also include housing loops 213 to allow for better grip, including for one handed operation of the device. The device 200 may also include an insulating sheath 215 on the electrode needle 221 that can be deployed and retracted to adjust the treatment field for the individual patient and their anatomy. The housing may be operatively connected to a penetration limiting means 214 that ensures that the electrodes needle 221 does not exceed a predetermined maximum depth of penetration.

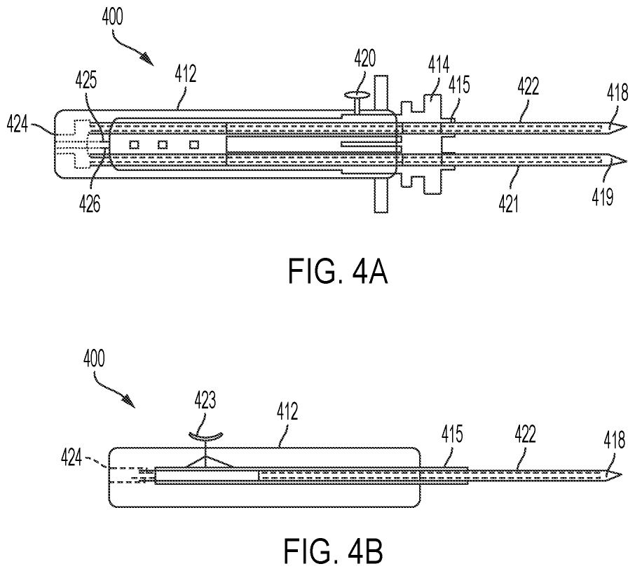

Referring to FIG. 3, the in vivo electroporation device 300 may comprise a housing 312 comprising two electrode needles 322. The electrode needles each comprise a hollow shaft which includes an infusion chamber therein (not shown), a penetrating tip 318, and a plurality of infusion ports 319 along an axially extending length of the shaft of the electrode needle 322. The infusion ports on one electrode needle are shown facing the infusion ports on the other electrode needle. The electrode needle is configured to administer the therapeutic substance through the plurality of infusion ports 319. The housing 312 may include a handle 323 configured to slide the insulating sheath 315 on the electrode needle 322 into position. The housing may be operatively connected to a penetration limiting means 314 that ensures that the electrode needles 322 do not exceed a predetermined maximum depth of penetration. The housing may further include a penetration limiting lock 320 which sets the penetration limiting means 314 at a predetermined maximum depth, protecting vital internal structures. The penetration limiting means 314 is situated on the housing so that as it slides it doesn't impede the insulating sheath 315.

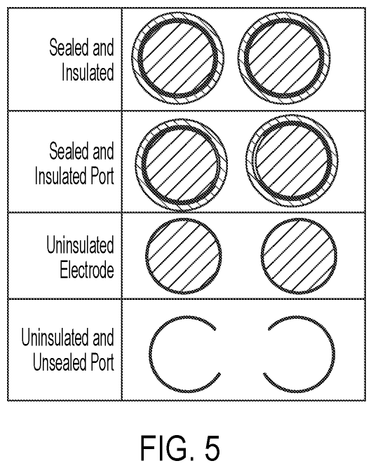

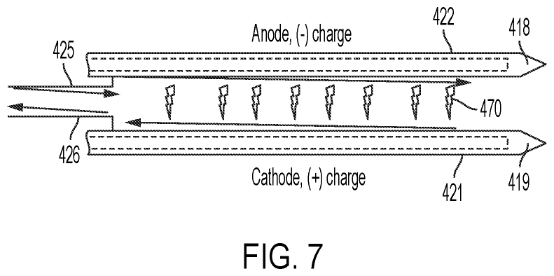

Referring to FIG. 4A and 4B, the in vivo electroporation device 400 may comprise a housing 412 comprising a electrode needle 421, which is shown as a charged cathode electrode, and a second electrode needle 422, which is shown as a charged anode electrode,. The electrode needles 421, 422 each comprise a hollow shaft which includes an infusion chamber (not shown), penetrating tips 418, 419. The housing 412 may include a handle 423 configured to slide the insulating sheath 415 on the electrode needles 421, 422 into position. The housing may be operatively connected to a penetration limiting means 414 that ensures that the electrode needles 421, 422 do not exceed a predetermined maximum depth of penetration. The housing may further include a penetration limiting lock 420 which sets the penetration limiting means 414 at a predetermined maximum depth, protecting vital internal structures. The therapeutic substance may be delivered from a fluid delivery means such as an infusion pump which is operatively coupled to the device through an infusion pump port 424 on proximal end of the housing for coupling with the infusion pump. The infusion pump port 424 may bifurcate and be operatively coupled to an infusion chamber in each electrode needles 421, 422. The charged anode electrode may be connected to the device at the anode conductive attachment bracket 425 (also shown in FIG. 7) and the cathode electrode may be connected to the device at the cathode conductive attachment bracket 426. The conductive brackets of each of the electrodes (i.e. anode and/or cathode) may be in direct contact with the conductive materials of the electrode. It is believed that direct contact allows efficient circuit movement of the electrical current from the generator through the electrodes and back to the cathode cord. Attachment of the conductive element (e.g. wire) from the generator to the bracket may include a means of pinching the conductive element securely to the conductive material of the bracket ensuring sufficient surface contact for reliable and consistent flow of current. The attachment may include but is not limited to screwing the conductive element from the generator to the bracket, pinching the conductive element between two plates of the bracket, and/or wrapping the conductive element around the bracket. The conductive element may comprise a wire, a conductive filler, coating, a combination thereof, or the like.

The housing may further include an anode attachment bracket and a cathode attachment bracket to attach the respective electrode. Referring to FIG. 7, there is a conductive anode attachment bracket 425 at the proximal end of the electrode needle 422, which is in direct contact with the conductive materials therein and thus functionalizing it as a charged anode electrode. Additionally, there is a conductive cathode attachment bracket 426 at the proximal end of the electrode needle 421, which is in direct contact with the conductive material therein and thus functionalizing it as a charged cathode electrode. When activated, the anode conductive bracket transfers the current from a cord connected to the anode port of an external power source to the monopolar anode electrode needle. Then, as shown in FIG. 7, an electric current 470 flows through the gap/tissue between the electrode needle 422 (operating at the negative, anode electrode) and the electrode needle 421 (operating as the positive, cathode electrode) creating a voltage potential between the two needles. The voltage potential provides a pulse of electrical energy to the at least two electrode needles causing poration of the target tissue's cells for their uptake of the therapeutic substance. Subsequently, current flows down the electrode needle 421 to the cathode bracket 426 which transfers the current to the cord attached to a cathode port of the power source (not shown).

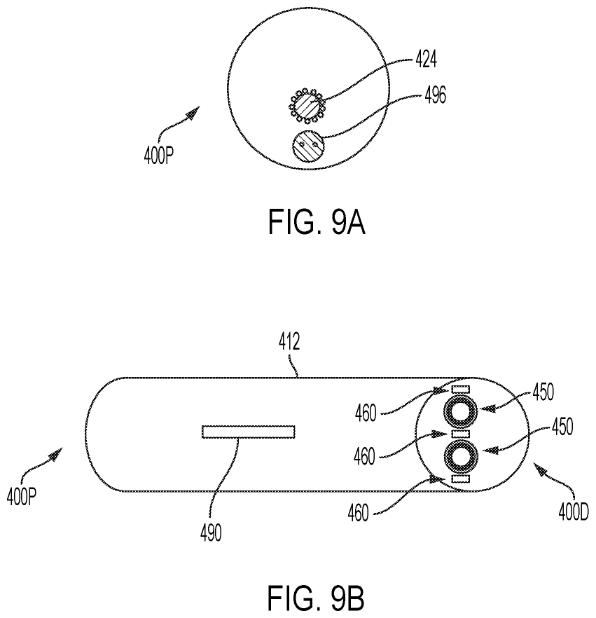

Referring to FIG. 9A and 9B, the housing 412 of the device may comprise a rigid ergonomic material that fits easily in the hand. There may be a slit shaped opening 490 on the top of the housing for the handle of the insulating sheath. At the proximal end 400P of the housing as shown in FIG. 9A, the housing may include an infusion pump port 424 for attaching tubing and/or a fluid delivery means, such as an infusion pump. Below the infusion port, the housing may include a plug-in 496 for an electrical connection to an external power source. The electrical connection may connect the device's electrical circuit to the electrical current generator. At the distal end 400D of the housing as shown in FIG. 9B, there are openings 450 for the electrode needles, the insulating sheath, and slides 460 for the penetration limiting means. Optional handle loops or wings may also be included to increase device maneuverability.

At least a portion of the infusion chamber, optionally located within the shaft, may comprise an insulating material. The insulating material may include vinyl/polyvinyl chloride, red rubber latex, silicone rubber polymer, a combination thereof, or the like.

When there are a plurality of electrode needles, they may be the same in length. The electrode needle(s) may be the same length as the injection needle (if separate), or they may be different lengths. The length of the electrode needle in total may be about 200 mm to about 450 mm, about 210 mm to about 450 mm, about 250 mm to about 450 mm, about 280 mm to about 450 mm, about 300 mm to about 450 mm, about 320 mm to about 450 mm, about 350 mm to about 450 mm, about 380 mm to about 450 mm, about 200 mm to about 420 mm, about 200 mm to about 400 mm, about 200 mm to about 380 mm, about 200 mm to about 350 mm, about 200 mm to about 320 mm, about 200 mm to about 300 mm, or a range between any two of these values. At least a part of the electrode needle may be situated in the housing and at least part of the electrode needle may extend out from the housing. About 80 mm to about 250 mm in length of the electrode needle may be within the housing. About 120 mm to about 200 mm, or about 150 mm to about 180 mm in length of the electrode needle may extend outside of the housing. As an example, the electrode needle may be about 210 mm in length within the housing and 170 mm of the electrode needle extending out from the housing. As another example, the electrode needle may be about 274 mm in length, with 105 mm of the electrode needle within the housing and 170 mm of the electrode needle extending out from the housing.

The treatment field may be pulsed after a waiting period. For example, the treatment field may be pulsed up to 10 minutes after administering the therapeutic substance. The treatment field may be pulsed up to about 8 minutes, up to about 6 minutes, up to about 5 minutes, up to about 3 minutes, about 1 minute to about 10 minutes, about 1 minute to about 8 minutes, about 1 minute to about 5 minutes, about 3 minutes to about 10 minutes, about 3 minutes to about 8 minutes, or about 3 minutes to about 5 minutes, after administering the therapeutic substance.

The in vivo electroporation device may be a gene therapy electroporation device, a chemo-electroporation device, or a device for immune system sensitization. The therapeutic substance may include genetic material, a pharmaceutical agent, a biologic, a chemical drug, or a combination thereof.

As used herein, the term “tissue” refers to any aggregation of similarly specialized mammalian cells which are united in the performance of a particular function, such as human tissue. The tissue may be any human tissue, such as, without limitation, soft tissue, fatty tissue, muscle tissue, bone tissue, serosa, connective tissue, or tissue from organs, such as heart, liver, lungs, brain, etc. For example, the tissue may include skin, subcutaneous tissue, intradermal tissue, subdermal tissue, skeletal muscle, striated muscle, smooth muscle, organs, heart, breast, lung, pancreas, liver, spleen, mucosa, or a combination thereof. For example, the device may be used to target the liver for delivery of gene therapy. The device may be used for percutaneous liver procedures. Target tissues may include both healthy and diseased cells. The technique can also be utilized for application in healthy or diseased organs that must be accessed via minimally invasive or other surgical means. Such target tissues include the liver, lungs, heart, blood vessels, lymphatic, brain, kidneys, pancreas, stomach, intestines, colon, bladder, and reproductive organs. One should note that the desired therapeutic effect may be derived from agent delivery to cell types normally located within the target tissues as well as other cell types abnormally found within said tissues (e.g. chemotherapeutic treatment of tumors).

The device disclosed herein may be used to deliver electric pulse and a therapeutic substance directly into the tissue parenchyma. Rather than using the vasculature to access target tissue, the device disclosed in embodiments herein may be used to directly target tissue, such as the liver, heart, deep muscle, kidney, pancreas, breast, lung, spleen, skin, or other such tissues.

The in vivo electroporation device may further comprise a reservoir having an adjustable volume in fluid communication with the shaft. The reservoir may include, for example, a syringe, such as spring-type and vacuum-type syringe pumps, controlled motor pumps, and elastomeric/balloon pump, roller peristaltic pumps, piston pumps or the like.

The reservoir may have a variable volume capacity of up to about 10 ml. The reservoir may have a variable volume capacity of up to about 5 ml, greater than 0.0 ml to about 10 ml, greater than 0.0 ml to about 5 ml, greater than 0.0 ml to about 1 ml, greater than 0.0 ml to about 0.5 ml, or the like.

The in vivo electroporation device may include a means for generating an electrical signal operatively connected to the electrodes. The means for generating an electrical signal may include a wave form generator utilizing AC sine wave, square waves, and/or exponential decay waves, an electroporation pulse generator, electrofusion system, transfection system, or any electrical signal generator.

The method of using a device described herein may further include applying the electrical signal to the penetrating electrode needles such that resulting electric field propagation in said predetermined tissue site is of sufficient magnitude and duration to increase the intracellular uptake of said therapeutic substance.

The electrical signal may include at least one monopolar direct current pulse. At least one of said monopolar direct current pulse may have a duration of 0.1 milliseconds to 100 milliseconds and an amplitude capable of inducing an electrical field of from approximately 50 to approximately 300 V/cm between the plurality of electrode needles. The pulse length could range from 10 μsec to 99 ms depending on application, and whether the objective is gene therapy, tumor eradication, or a different clinical application.

The electrode needles may include conductive elements whose size and shape are sufficient to enable insertion through the matter covering a tissue of interest, such as skin covering muscle tissue, or the outer layer(s) of such tissue. For example, the electrode needle may include a penetration tip which pierces through layers of tissue. The electrode needle may comprise penetrating electrode array systems, such as bipolar and multielement electrode arrays. For example, the electrode needle may be arranged in a bipolar array system, including two penetrating electrodes connected to the opposite poles of a pulse generator.

The in vivo electroporation device may include a fluid delivery means. The fluid delivery means may comprise a piston adapted to inject fluid at a controlled rate, such as without limitation a spring-type syringe pump, a vacuum-type syringe pump, a controlled motor pump, an elastomeric/balloon pump, roller peristaltic pump, or other such pumps. The electrode needle may also comprise infusion ports and a hollow shaft including an infusion chamber to allow for delivery of the therapeutic substance. In an embodiment, the plurality of infusion ports on an axially extending line of the at least one electrode needle may face the plurality of infusion ports on an axially extending line of another, second electrode needle.

In certain embodiments, the electrode needle may function as an injection needle and as an electrode needle. Electrodes may be integrated into the device either as part of the injection needle(s), or as separate electrode needle(s). The needles may be configured in a fixed array allowing excellent profusion and minimal tissue damage. The injection needle will be either centrally located within the electrical field with numerous infusion ports allowing steady infusion of the treatment along the entire length of the needle, or the injection needle will be integrated into the electrode probes so that a single hollow penetrating device can both house the electrode and inject the infusion, allowing for fewer physical tissue piercing sites and a predictable infusion pattern. The injection needle may comprise a plurality of infusion ports to allow for delivery of the infusion.

The device may include a retractable insulating and sealing sheath that may be extended over the electrode needle to enlarge or shrink the treatment field based on the needs and anatomy of each individual patient. The electrical field and infusion field may extend along a large portion of the length of the needle(s) allowing a large area to be transduced at once.

An in vivo electroporation device may include a housing. The housing includes: a) an injection needle comprising a penetration tip; and a shaft that is elongated, hollow, and adapted for delivery of a therapeutic substance to a tissue, said shaft having a length running from a proximal end of the injection needle to the tip of the injection needle at a distal end of the needle, wherein the shaft comprises a plurality of infusion ports; b) at least two electrode needles, each electrode needle comprising a penetration tip, a shaft having a length running from a proximal end of the electrode needle to the tip of the electrode needle at a distal end of the needle and comprising at least one electrode adapted to produce an electric field, and an insulating sheath surrounding the shaft and configured to move relative to the shaft between an extended position covering the distal end of the shaft and a retracted position exposing the distal end of the shaft; and c) a penetration limiting means for controlling the depth of insertion of the at least two electrode needles and the injection needle.

The electrode needle may end near the proximal aspect of the outer housing and a bifurcated connector may seal over the end of each hollow electrode needle. The cathode and anode connecter plate may be just distal to the bifurcated connector attachment. The bifurcated connector may comprise an insulated material, optionally the same material as the sheath. The bifurcated connecter may connect the infusion port to the hollow electrode needles.

In an embodiment, a handheld in vivo electroporation device may comprise a bipolar electrode needle having a hollow center with a plurality of infusion ports along both sides of the shaft. The device may further include an insulating sheath that can be deployed and retracted to adjust the treatment field to each individual patient and their anatomy. The device may allow for single hand operation, and may, for example, allow the other hand to simultaneously operate the ultrasound probe for precise placement of the needle(s).

A method of treating a disease in a patient in need thereof is disclosed, comprising inserting a device disclosed herein having a therapeutic substance disposed therein into a tissue of the patient in vivo. The device may include a housing comprising at least one electrode needle comprising a penetration tip; and a shaft that is elongated, hollow, and adapted for delivery of the therapeutic substance to a tissue, said shaft having a length running from one end of the needle to the penetration tip of the needle at the other end of the needle, wherein the shaft comprises a charged electrode adapted to produce an electric field, and a plurality of infusion ports on at least one axially extending line along the length, a retractable insulating sheath configured to limit a treatment field of the device, and a penetration limiting means configured to limit the depth of insertion of the electrode needle, and activating an electrical energy source in electrical communication with each of the at least one electrode needle, thereby providing a pulse of electrical energy to the at least one electrode causing poration of the target tissue's cells for their uptake of the therapeutic substance. The method may include setting the penetration limiting means on the device to a maximum depth of insertion before insertion of the device into the patient. The method may include extending an insulating sheath of the device over the at least one electrode to reduce the size of a therapeutic field before activating an electrical energy source.

In an example embodiment, a method of treating a disease in a patient in need thereof, the method comprising inserting a device comprising a therapeutic substance disposed therein into a tissue of the patient in vivo, wherein the device further comprises a housing comprising an injection needle comprising a penetration tip; and a shaft that is elongated, hollow, and adapted for delivery of a therapeutic substance to a tissue, said shaft having a length running from a distal end of the injection needle to the tip of the injection needle at a proximal end of the needle, wherein the shaft comprises a plurality of infusion ports, at least two electrode needles, each electrode needle comprising a penetration tip, a shaft having a length running from a distal end of the electrode needle to the tip of the electrode needle at a proximal end of the needle and comprising at least one electrode adapted to produce an electric field; and an insulating sheath surrounding the shaft and configured to move relative to the shaft between an extended position covering the distal end of the shaft and a retracted position exposing the distal end of the shaft, and a penetration limiting means for controlling the depth of insertion of the at least two electrode needles and the injection needle; and activating an electrical energy source in electrical communication with each of the at least two electrode needles, thereby providing a pulse of electrical energy to the at least two electrode causing poration of the target tissue's cells for their uptake of the therapeutic substance.

Given that multiple applications using the device disclosed in embodiments herein may be contemplated and that there are significant differences in body and tissue composition between patients, it is likely that there may be a need to adjust the injection parameters. Such injection parameters include the needle size, injectant viscosity, injectant volume, concentration of therapeutics, and injection rate. Adjusting these parameters may be desirable to compensate for various characteristics of the therapy recipient, including age, weight, dosing, target tissue type, and the target tissue depth, especially for transcutaneous administration (which may be affected, among other factors, by the recipient's age and level of obesity).

It must also be noted that as used in the specification and in the appended claims, the singular forms “a”, “an”, and “the” include plural references unless the context clearly dictates otherwise. Thus, for example, reference to “a colorant” is a reference to one or more colorants and equivalents thereof known to those skilled in the art, and so forth. Also, the terms “includes” and/or “including,” when used in this specification, specify the presence of stated features, elements, and/or components, but do not preclude the presence or addition of one or more other features, steps, operations, elements, components, and/or groups thereof.

As used herein, the term “about” means plus or minus 10% of the numerical value of the number with which it is being used. Therefore, about 50% means in the range of 45%-55%.

As used herein, the term “optional” or “optionally” means that the subsequently described event or circumstance may or may not occur, and that the description includes instances where the event occurs and instances where it does not.

As used herein, the term “room temperature” means an indoor temperature of from about 20° C. to about 25° C. (68 to 77° F.).

Throughout the specification of the application, various terms are used such as “primary,” “secondary,” “first,” “second,” and the like. These terms are words of convenience in order to distinguish between different elements, and such terms are not intended to be limiting as to how the different elements may be utilized.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

What is claimed:

1. An in vivo electroporation device comprising:
a housing comprising
at least one electrode needle comprising:
a penetration tip; and
a shaft that is elongated, hollow, and adapted for delivery of a therapeutic substance to a tissue, said shaft having a length running from one end of the needle to the penetration tip of the needle at the other end of the needle, wherein the shaft comprises
a charged electrode adapted to produce an electric field; and
a plurality of infusion ports on at least one axially extending line along the length of the electrode needle;
a retractable insulating sheath configured to limit a treatment field of the device; and
a penetration limiting means configured to limit the depth of insertion of the electrode needle.

2. The device of claim 1, wherein the charged electrode comprises at least two monopolar electrodes, and wherein the at least two monopolar electrodes are spaced and electrically isolated from one another.

3. The device of claim 1 or 2, wherein the charged electrode comprises a bipolar electrode.

4. The device of any of the preceding claims, wherein the charged electrode runs an entire length of the shaft.

5. The device of any of the preceding claims, wherein the device is a gene therapy electroporation device, a chemo-electroporation device, or a device for immune system sensitization.

6. The device of any of the preceding claims, wherein the therapeutic substance comprises genetic material, a pharmaceutical agent, a biologic, a chemical drug, or a combination thereof.

7. The device of any of the preceding claims, wherein the tissue comprises skin, subcutaneous tissue, intradermal tissue, subdermal tissue, skeletal muscle, striated muscle, smooth muscle, organs, heart, breast, lung, pancreas, liver, spleen, mucosa, or a combination thereof.

8. The device according to any of the preceding claims further comprising a reservoir having an adjustable volume in fluid communication with the shaft.

9. The device according to claim 8, wherein the reservoir comprises a syringe.

10. The device according to claim 8 or 9, wherein the reservoir has a variable volume capacity of greater than 0.0 ml to about 5 ml.

11. The device according to any of the preceding claims further comprising a means for generating an electrical signal operatively connected to the electrodes.

12. The device of claim 11, wherein the means for generating the electrical signal comprises an electroporation pulse generator.

13. The device of any of the preceding claims, further comprising a fluid delivery means.

14. The device of claim 13, wherein the fluid delivery means comprises a piston adapted to inject fluid at a controlled rate.

15. The device of any of the preceding claims, wherein the plurality of infusion ports on an axially extending line of the at least one electrode needle faces a plurality of infusion ports on an axially extending line of a second electrode needle.

16. An in vivo electroporation device comprising:
a housing comprising
an injection needle comprising:
a penetration tip; and
a shaft that is elongated, hollow, and adapted for delivery of a therapeutic substance to a tissue, said shaft having a length running from a distal end of the injection needle to the tip of the injection needle at a proximal end of the needle, wherein the shaft comprises a plurality of infusion ports;
at least two electrode needles, each electrode needle comprising:
a penetration tip;
a shaft having a length running from a distal end of the electrode needle to the tip of the electrode needle at a proximal end of the needle and comprising at least one electrode adapted to produce an electric field; and
an insulating sheath surrounding the shaft and configured to move relative to the shaft between an extended position covering the distal end of the shaft and a retracted position exposing the distal end of the shaft; and
a penetration limiting means for controlling the depth of insertion of the at least two electrode needles and the injection needle.

17. The device according to claim 16 further comprising a reservoir having an adjustable volume in fluid communication with said shaft.

18. The device according to claim 17, wherein the reservoir comprises a syringe.

19. The device according to claim 17 or 18, wherein the reservoir has a variable volume capacity of greater than 0.0 ml to about 5 ml.

20. The device according to any of claims 16 to 19 further comprising a means for generating an electrical signal operatively connected to the at least two electrodes.

21. The device of claim 20, wherein the means for generating an electrical signal comprises an electroporation pulse generator.

22. The device of any of claims 16 to 21 further comprising:
a fluid delivery means comprising
an infusion pump attached to a proximal end of the housing;
an infusion chamber within the housing coupled to the infusion pump;
a plurality of infusion ports on the injection needle adapted for delivery of fluids coupled to the infusion chamber.

23. The device of claim 22, wherein the fluid delivery means comprises a piston adapted to inject fluid at a controlled rate.

24. The device of any of claims 16 to 23, wherein each of the at least two electrodes comprises a monopolar electrode.

25. The device of any of claims 16 to 24, wherein each of the at least two electrodes comprises a bipolar electrode.

26. The device of any of claims 16 to 25, wherein each of the at least two electrodes runs an entire length of the shaft.

27. The device of any of claims 16 to 26, wherein the device is a gene therapy electroporation device, a chemo-electroporation device, or a device for immune system sensitization.

28. The device of any of claims 16 to 27, wherein the therapeutic substance comprises genetic material, a pharmaceutical agent, a biologic, a chemical drug, or a combination thereof.

29. The device of any of claims 16 to 28, wherein the tissue comprises skin, subcutaneous tissue, intradermal tissue, subdermal tissue, skeletal muscle, striated muscle, smooth muscle, organs, heart, breast, lung, pancreas, liver, spleen, mucosa, or a combination thereof.

30. A method of treating a disease in a patient in need thereof, the method comprising:
inserting the device of any one of claims 1-15 containing a therapeutic substance disposed therein into a tissue of the patient in vivo, and
activating an electrical energy source in electrical communication with each of the at least one electrode needle, thereby providing a pulse of electrical energy to the at least one electrode needle causing poration of the target tissue's cells for uptake of the therapeutic substance.

31. The method of claim 30 further comprising setting the penetration limiting means on the device to a maximum depth of insertion before insertion of the device into the patient.

32. The method of claim 30 or 31 further comprising extending an insulating sheath of the device over the at least one electrode to reduce the size of a therapeutic field before activating an electrical energy source.

33. The method of any of claims 30 to 32, wherein the at least one electrode is disposed within the hollow shaft of the injection needle.

34. The method of any of claims 30 to 33, wherein the at least one electrode is disposed within a hollow shaft of an electrode needle on the device.

35. The method of any of claims 30 to 34, wherein the therapeutic substance comprises genetic material, a pharmaceutical agent, a biologic, a chemical drug, or a combination thereof.

36. The method of any of claims 30 to 35, wherein the tissue comprises skin, subcutaneous tissue, intradermal tissue, subdermal tissue, skeletal muscle, striated muscle, smooth muscle, organs, heart, breast, lung, pancreas, liver, spleen, mucosa, or a combination thereof.

37. The method of any of claims 30 to 36, wherein the therapeutic substance is disposed in a reservoir of the device, wherein the reservoir has an adjustable volume in fluid communication with the hollow shaft of the injection needle.

38. A method of treating a disease in a patient in need thereof, the method comprising:
inserting the device of any one of claims 16-29 containing a therapeutic substance disposed therein into a tissue of the patient in vivo, and
activating an electrical energy source in electrical communication with each of the at least two electrode needles, thereby providing a pulse of electrical energy to the at least two electrode needles causing poration of the target tissue's cells for their uptake of the therapeutic substance.
